(19) United States
(12) Patent Application Publication
(10) Pub. No.: US 2019/0183726 A1
(43) Pub. Date: Jun. 20, 2019

(54) DETERMINATION OF CARDIOPULMONARY RESUSCITATION COMPRESSION RATE

(71) Applicant: HeartSine Technologies Limited, Belfast (GB)

(72) Inventors: Paul McCanny, Newtownabbey (GB); Ian Todd, Newcastle (GB)

(21) Appl. No.: 16/219,271

(22) Filed: Dec. 13, 2018

(30) Foreign Application Priority Data
Dec. 19, 2017 (GB) 1721255.6

(51) Int. Cl. A61H 31/00; A61N 1/39

(52) U.S. Cl. CPC A61H 31/005; A61N 1/39044; A61H 2201/5061; A61H 31/007; A61H 2201/5071; A61H 2201/5084

(57) ABSTRACT

A defibrillator for determining a cardiopulmonary resuscitation (CPR) compression rate, including electrodes adapted to be attached to the subject, an impedance signal measurement system connected to the electrodes and configured to measure at least one impedance signal of the subject, an electrocardiogram signal measurement system connected to the electrodes, an impedance signal processing system connected to the impedance signal measurement system, an electrocardiogram signal processing system connected to the electrocardiogram signal measurement system, a compression rate estimate processing system configured to apply a plurality of criteria to the impedance signal features and the electrocardiogram signal features and use compliance with one or more of the criteria to select one of the plurality of impedance signal compression rate estimates as the cardiopulmonary resuscitation compression rate, and an output unit configured to output feedback based on the cardiopulmonary resuscitation compression rate to a user of the defibrillator.

### PRIORITY INFORMATION

The present application claims priority of from UK patent application No. 1721255.6, filed 19 Dec. 2017, the content of which is incorporated herein by reference in its entirety.

### BACKGROUND OF THE INVENTION

### 1. Field of the Invention

The disclosure relates to the determination of cardiopulmonary resuscitation compression rate, particularly in real-time using a defibrillator.

### 2. Introduction

When using a defibrillator on a subject, the defibrillator will often advise the user to perform cardiopulmonary resuscitation (CPR) on the subject. CPR is performed to ensure that oxygenated blood remains circulating in the body of the subject in the absence of a heartbeat. CPR may be advised, for example, between application of one or more defibrillation shocks to the subject or when the subject's electrocardiogram (ECG) indicates that CPR, rather than defibrillation shocks, is the best treatment. Performance of effective CPR has been shown to increase a subject's chance of survival. To be effective, CPR compressions must be performed at a rate suitable for the subject and rate determination of the CPR compressions is therefore important.

CPR compressions involve the compression and release of the chest of the subject. Commonly, accelerometers are employed with defibrillators to directly measure the motion of the chest for CPR compression rate determination. However, there are problems associated with this rate determination method. As accelerometers measure chest motion by double integration of the measured acceleration of the chest, compression rate determination from the measured chest acceleration can include large uncertainties and may not be accurate. In a static environment, this problem may be limited, but in a real-world environment there may be motion of the subject which could lead to issues with rate determination. Accelerometers may also be subject to noisy signal inputs if they move relative to the body of the subject, further complicating CPR compression rate determination. In addition, the use of an additional sensor with the defibrillator increases the complexity of the defibrillator.

Pressure sensitive pads may be employed with defibrillators to measure the compressions applied to the subject's chest. These are also subject to issues with noise and the complication, cost and size of additional sensors.

### SUMMARY

Additional features and advantages of the disclosure will be set forth in the description which follows, and in part will be obvious from the description, or may be learned by practice of the disclosure. The features and advantages of the disclosure may be realized and obtained by means of the instruments and combinations particularly pointed out in the appended claims. These and other features of the present disclosure will become more fully apparent from the following description and appended claims, or may be learned by the practice of the disclosure as set forth herein.

The following concepts address the issues in the art identified above. Chest compressions performed during CPR produce changes to transthoracic impedance of the subject. The subject's chest acts as a resistor to an applied electrical current and when the volume of the chest changes due to CPR compressions, the transthoracic impedance will change. Transthoracic impedance variations may therefore be used to determine the rate of compressions. This rate determination method also has associated problems. The morphology of the impedance signal may change over time. This may be due to the subject's chest shape physically changing as a result of compressions, or perhaps because a different person is administering CPR, or the person administering CPR is fatigued. The reality of rescue of a subject in the field means moving subjects and compressions that vary in rate, force and application angle in a short space of time. The impedance signal morphology may change in such a way as to confuse conventional compression rate determination methods.

According to a first aspect of the disclosure there is provided a defibrillator for determining a rate of cardiopulmonary resuscitation compressions on a subject, including one or more of electrodes adapted to be attached to the subject, an impedance signal measurement system connected to the electrodes and configured to measure at least one impedance signal of the subject, an electrocardiogram signal measurement system connected to the electrodes and configured to measure at least one electrocardiogram signal of the subject, an impedance signal processing system connected to the impedance signal measurement system and configured to process the impedance signal of the subject to obtain a plurality of impedance signal compression rate estimates and a plurality of impedance signal features, an electrocardiogram signal processing system connected to the electrocardiogram signal measurement system and configured to process the electrocardiogram signal of the subject to obtain a plurality of electrocardiogram signal features, a compression rate estimate processing system connected to the impedance signal measurement system and the electrocardiogram signal measurement system and configured to apply a plurality of criteria to the impedance signal features and the electrocardiogram signal features and use compliance with one or more of the criteria to select one of the plurality of impedance signal compression rate estimates as the cardiopulmonary resuscitation compression rate, and an output unit connected to the compression rate estimate processing system and configured to output feedback based on the cardiopulmonary resuscitation compression rate to a user of the defibrillator. Any combination of these features can apply.

The impedance signal measurement system may be configured to measure the at least one impedance signal by continuously sampling impedance data received from the electrodes. The ECG signal measurement system can be configured to measure the at least one ECG signal by continuously sampling ECG data received from the electrodes.

Processing the impedance signal of the subject to obtain the plurality of impedance signal compression rate estimates can include one or more of using a frequency domain transformation on the impedance signal to obtain an impedance signal frequency spectrum, using a peak detection algorithm to identify a plurality of peaks in the impedance signal frequency spectrum and/or determining central frequencies of the plurality of peaks as the plurality of impedance signal compression rate estimates.

Processing the impedance signal of the subject to obtain the plurality of impedance signal features can include one or more of using a frequency domain transformation on the impedance signal to obtain an impedance signal frequency spectrum, using a peak detection algorithm to identify a plurality of peaks in the impedance signal frequency spectrum and/or determining central frequencies and amplitudes of the plurality of peaks as the plurality of impedance signal features.

Processing the ECG signal of the subject to obtain the plurality of ECG signal features can include one or more of using a frequency domain transformation on the ECG signal to obtain an ECG signal frequency spectrum, using a peak detection algorithm to identify a plurality of peaks in the ECG signal frequency spectrum and/or determining central frequencies and amplitudes of the plurality of peaks as the plurality of ECG signal features.

Using the frequency domain transformation on the impedance signal to obtain the impedance signal frequency spectrum can include using a Fast Fourier Transform (FFT) algorithm on a window of the impedance signal. Using the frequency domain transformation on the impedance signal to obtain the impedance signal frequency spectrum can include using a Goertzel algorithm on a window of the impedance signal. It will be appreciated that other frequency domain transformation methods may be used.

Using the frequency domain transformation on the ECG signal to obtain the ECG signal frequency spectrum can include using a FFT algorithm on a window of the ECG signal corresponding to the window of the impedance signal. Using the frequency domain transformation on the ECG signal to obtain the ECG signal frequency spectrum can include using a Goertzel algorithm on a window of the ECG signal corresponding to the window of the impedance signal. It will be appreciated that other frequency domain transformation methods may be used.

The window of the impedance signal and the ECG signal can include a window size in the range of 3 seconds to 20 seconds. The window can include a six second window size. The window may be an advancing window. The window may advance by a period which is less than the window size. The window may advance by a period of 1 second or other period of time.

Using the peak detection algorithm to identify a plurality of peaks in the impedance signal frequency spectrum can include identifying peaks based on decreasing steepness of slopes of the impedance signal frequency spectrum. Using the peak detection algorithm to identify a plurality of peaks in the ECG signal frequency spectrum can include identifying peaks based on decreasing steepness of slopes of the ECG signal frequency spectrum. It will be appreciated that some other methods of peak detection may be used. The peak detection algorithm may return a primary peak having a highest amplitude at its central frequency, a secondary peak having a next highest amplitude at its central frequency continued up to a specified number of peaks. The peak detection algorithm may use an amplitude threshold to determine if a peak is a true peak. The amplitude threshold can include a pre-determined number of standard deviations above background.

The impedance signal processing system may process the impedance signal to obtain a plurality of additional impedance signal features from the impedance signal including any one or more of variance of the impedance signal, morphology of the impedance signal, gradient of the impedance signal, power of the impedance signal, wavelet decomposition of the impedance signal, noise analysis of the impedance signal, cepstrum of the impedance signal.

The ECG signal processing system may process the ECG signal to obtain a plurality of additional ECG signal features from the ECG signal including any one or more of variance of the ECG signal, morphology of the ECG signal, gradient of the ECG signal, power of the ECG signal, wavelet decomposition of the ECG signal, noise analysis of the ECG signal, cepstrum of the ECG signal.

The plurality of criteria applied by the compression rate estimate processing system can include a criterion including in the impedance signal frequency spectrum a ratio of a secondary peak amplitude and a primary peak amplitude being greater than a pre-determined impedance signal amplitude ratio threshold. The impedance amplitude ratio threshold may be 0.2 or a value in a range from 0.05 to 0.5, as well as values outside of this range.

The plurality of criteria applied by the compression rate estimate processing system can include a criterion including in the impedance signal frequency spectrum a central frequency of a primary peak being greater than a central frequency of a secondary peak.

The plurality of criteria applied by the compression rate estimate processing system can include a criterion including in the ECG signal frequency spectrum a central frequency of a primary peak being less than a central frequency of a secondary peak.

The plurality of criteria applied by the compression rate estimate processing system can include a criterion including a frequency difference between a lower frequency primary or secondary peak in the impedance signal frequency spectrum and a lower frequency primary or secondary peak in the ECG signal frequency spectrum being less than a pre-determined frequency difference threshold. The frequency difference threshold may be 0.25 Hz or in a range from 0.05 to 0.5 Hz, as well as values outside of this range. This criterion investigates whether an ECG signal frequency spectrum peak and an impedance signal frequency spectrum peak are aligned within a pre-determined limit.

The compression rate estimate processing system may use compliance with one or more of the plurality of criteria to select one of the plurality of impedance signal compression rate estimates as the CPR compression rate. The compression rate estimate processing system may use compliance with each of the plurality of criteria to select an impedance signal compression rate estimate including a central frequency of a secondary peak of the impedance signal frequency spectrum as the CPR compression rate. The compression rate estimate processing system may use non-compliance with any of the plurality of criteria to select an impedance signal compression rate estimate including a central frequency of a primary peak of the impedance signal frequency spectrum as the CPR compression rate.

The plurality of criteria applied by the compression rate estimate processing system can include a criterion including in the ECG signal frequency spectrum a ratio of a secondary peak amplitude and a primary peak amplitude being greater than a pre-determined ECG signal amplitude ratio threshold. The ECG signal amplitude ratio threshold may be 0.2 or a range including 0.05 to 0.5, or other values as well.

The compression rate estimate processing system may use compliance with each of the plurality of criteria to select an impedance signal compression rate estimate including a central frequency of a secondary peak of the impedance signal frequency spectrum as the CPR compression rate. The compression rate estimate processing system may use non-compliance with any of the plurality of criteria to select an impedance signal compression rate estimate including a central frequency of a primary peak of the impedance signal frequency spectrum as the CPR compression rate.

The defibrillator may determine a CPR compression rate using substantially all of the measured impedance signal and substantially all of the measured ECG signal. The defibrillator may determine a CPR compression rate using one or more portions of the measured impedance signal and corresponding one or more portions of the measured ECG signal. The one or more of each portion of the measured impedance signal can include portions which have been pre-analysed for CPR compression rate, which compression rate has not exceeded a threshold used to determine if the rate is a true rate. Thus, the CPR compression rate may be determined for one or more portions of the impedance signal using traditional impedance signal analysis techniques alone, and the CPR compression rate may be determined for one or more portions of the impedance signal using the method of the disclosure which combines using the impedance signal with using the ECG signal for qualification of the CPR compression rate.

The defibrillator may use processing of the impedance signal and the ECG signal to determine features based on a morphology of the impedance signal. The defibrillator may use processing of the impedance signal and the ECG signal to determine motion of the subject.

The output unit may output feedback based on the CPR compression rate to the user of the defibrillator including an indication that the CPR compression rate is any of satisfactory, too slow, too fast. Outputting the feedback based on the CPR compression rate to the user of the defibrillator provides the user with an opportunity to improve the quality of CPR and gives an indication of the effectiveness of the compressions.

According to a second aspect of the disclosure there is provided a method of determining a cardiopulmonary resuscitation (CPR) compression rate. The method can include one or more of receiving an impedance signal of a subject, receiving an electrocardiogram (ECG) signal of the subject, processing the impedance signal to obtain a plurality of impedance signal compression rate estimates, processing the impedance signal to obtain one or more of a plurality of impedance signal features, processing the ECG signal to obtain one or more of a plurality of ECG signal features, and applying a plurality of criteria to the impedance signal features and the ECG signal features and using compliance or non-compliance with one or more of the criteria to select one of the plurality of impedance signal compression rate estimates as the CPR compression rate.

According to a third aspect of the disclosure there is provided a CPR compression rate determination computer program, tangibly embodied on a computer readable medium, the computer program including instructions for causing a computer to execute the method of determining a rate of CPR compressions according to the second aspect of the disclosure.

The aspects of the disclosure allow a more robust determination of CPR compression rate than was previously possible using the impedance alone, without the need for additional sensors and using signals that are already measured in defibrillators.

### BRIEF DESCRIPTION OF THE DRAWINGS

In order to describe the manner in which the above-recited and other advantages and features of the disclosure can be obtained, a more particular description of the disclosure briefly described above will be rendered by reference to specific embodiments thereof which are illustrated in the appended drawings. Understanding that these drawings depict only exemplary embodiments of the disclosure and are not therefore to be considered to be limiting of its scope, the disclosure will be described and explained with additional specificity and detail through the use of the accompanying drawings in which:

FIG. 1 is a schematic representation of a defibrillator for determining a rate of CPR compressions on a subject according to the first aspect of the disclosure;

FIG. 2 is a flow chart representation of a method of determining a CPR compression rate carried out by the defibrillator of FIG. 1; and

FIG. 3 is flow chart representation showing further details of the method of FIG. 2.

### DETAILED DESCRIPTION

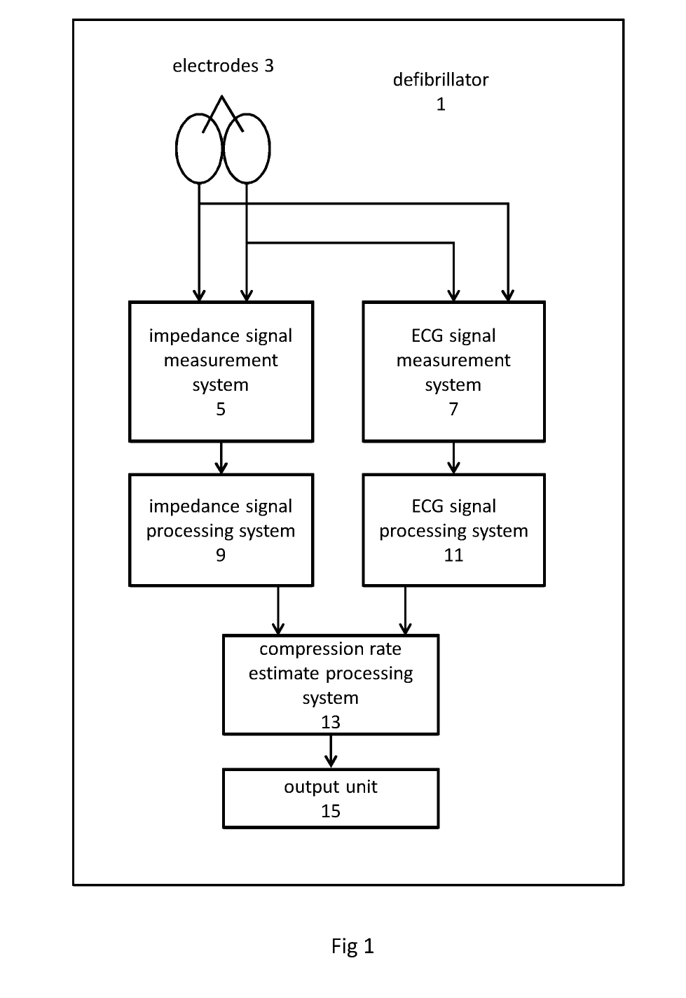

Referring to FIG. 1, a defibrillator 1 for determining a rate of CPR compressions on a subject is shown. This includes electrodes 32 adapted to be attached to the subject (not shown), an impedance signal measurement system 5 connected to the electrodes 3 and an ECG signal measurement system 7 connected to the electrodes 3. The defibrillator 1 further includes an impedance signal processing system 9 connected to the impedance signal measurement system 5, an ECG signal processing system 11 connected to the ECG signal measurement system 7, a compression rate estimate processing system 13 connected to the impedance signal processing system 9 and the ECG signal processing system 11 and an output unit 15 connected to the compression rate estimate processing system 13.

It will be appreciated that the defibrillator 1 will include other elements such as defibrillation shock generation circuitry, a power source.

The impedance signal measurement system 5 includes an impedance measurement signal generator (not shown) configured to generate an ac signal at a pre-determined voltage and an impedance measurement signal processor (not shown) including at least an amplifier module and a signal conditioning module (not shown). The impedance measurement signal generator generates the ac signal which is sent to the electrodes 3 and passes through the subject. The electrodes 3 produce impedance data indicative of the impedance of the subject which is passed to the impedance measurement signal processor. The impedance measurement signal processor continuously samples the impedance-indicative data received from the electrodes 3. The amplifier module and the signal conditioning module of the impedance measurement signal processor process the impedance data received from the electrodes 3 by any of amplification, filtering, analogue to digital conversion and signal processing. The impedance signal measurement system 5 thus measures at least one impedance signal of the subject which is passed to the impedance signal processing system 9.

The ECG signal measurement system 7 includes an ECG measurement signal processor (not shown). When placed on the subject, the electrodes 3 produce ECG data indicative of the ECG of the subject which is passed to the ECG measurement signal processor. The ECG measurement signal processor continuously samples the ECG-indicative data received from the electrodes 3 and then processes the ECG data received from the electrodes 3. The ECG signal measurement system 7 thus measures at least one ECG signal of the subject which is passed to the ECG signal processing system 11.

The impedance signal processing system 9 includes at least one micro processor which processes the impedance signal to obtain a plurality of impedance signal compression rate estimates and a plurality of impedance signal features. The ECG signal processing system 11 includes at least one micro processor which processes the ECG signal to obtain a plurality of ECG signal features. The compression rate estimate processing system 13 includes at least one micro processor which applies one or more of a plurality of criteria to the impedance signal features and the ECG signal features and uses compliance with one or more of the criteria to select one of the plurality of impedance signal compression rate estimates as the CPR compression rate. The output unit 15 receives the CPR compression rate and outputs feedback based on the CPR compression rate to a user of the defibrillator.

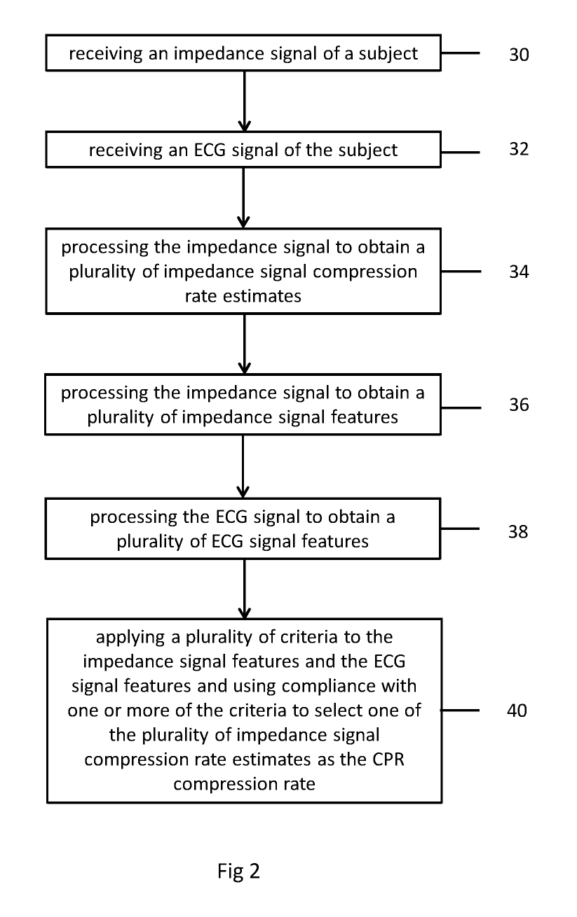

Referring to FIG. 2, the method of determining a CPR compression rate carried out by the defibrillator 1 of FIG. 1 includes one or more of: receiving an impedance signal of a subject (30), receiving an ECG signal of the subject (32), processing the impedance signal to obtain a plurality of impedance signal compression rate estimates (34), processing the impedance signal to obtain a plurality of impedance signal features (36), processing the ECG signal to obtain a plurality of ECG signal features (38), and applying a plurality of criteria to the impedance signal features and the ECG signal features and using compliance with one or more of the criteria to select one of the plurality of impedance signal compression rate estimates as the CPR compression rate (40).

Processing the impedance signal to obtain the plurality of impedance signal compression rate estimates 34 includes one or more of using a frequency domain transformation on the impedance signal to obtain an impedance signal frequency spectrum, using a peak detection algorithm to identify a plurality of peaks in the impedance signal frequency spectrum and determining central frequencies of the plurality of peaks as the plurality of impedance signal compression rate estimates.

Processing the impedance signal to obtain the plurality of impedance signal features 36 includes one or more of using a frequency domain transformation on the impedance signal to obtain an impedance signal frequency spectrum, using a peak detection algorithm to identify a plurality of peaks in the impedance signal frequency spectrum and determining central frequencies and amplitudes of the plurality of peaks as the plurality of impedance signal features. Processing the ECG signal to obtain the plurality of ECG signal features 38 includes using a frequency domain transformation on the ECG signal to obtain an ECG signal frequency spectrum, using a peak detection algorithm to identify a plurality of peaks in the ECG signal frequency spectrum and determining central frequencies and amplitudes of the plurality of peaks as the plurality of ECG signal features.

The transformation uses a FFT algorithm on a six second, advancing window of the impedance signal and a corresponding six second, advancing window of the ECG signal.

The peak detection algorithm identifies a plurality of peaks in the impedance signal frequency spectrum and ECG signal frequency spectrum by identifying peaks based on decreasing steepness of slopes of the spectra. In this embodiment, the peak detection algorithm returns two peaks from each frequency spectrum, a primary peak having a highest amplitude at its central frequency and a secondary peak having a next highest amplitude at its central frequency. An amplitude threshold, including a pre-determined number of standard deviations above background, is used to determine if the peaks are true peaks. The central frequencies of the primary peak and the secondary peak in the impedance signal frequency spectrum provide the impedance signal compression rate estimates. The central frequencies and amplitudes of the primary peak and the secondary peak in the impedance signal frequency spectrum provide the impedance signal features. The central frequencies and amplitudes of the primary peak and the secondary peak in the ECG signal frequency spectrum provide the ECG signal features.

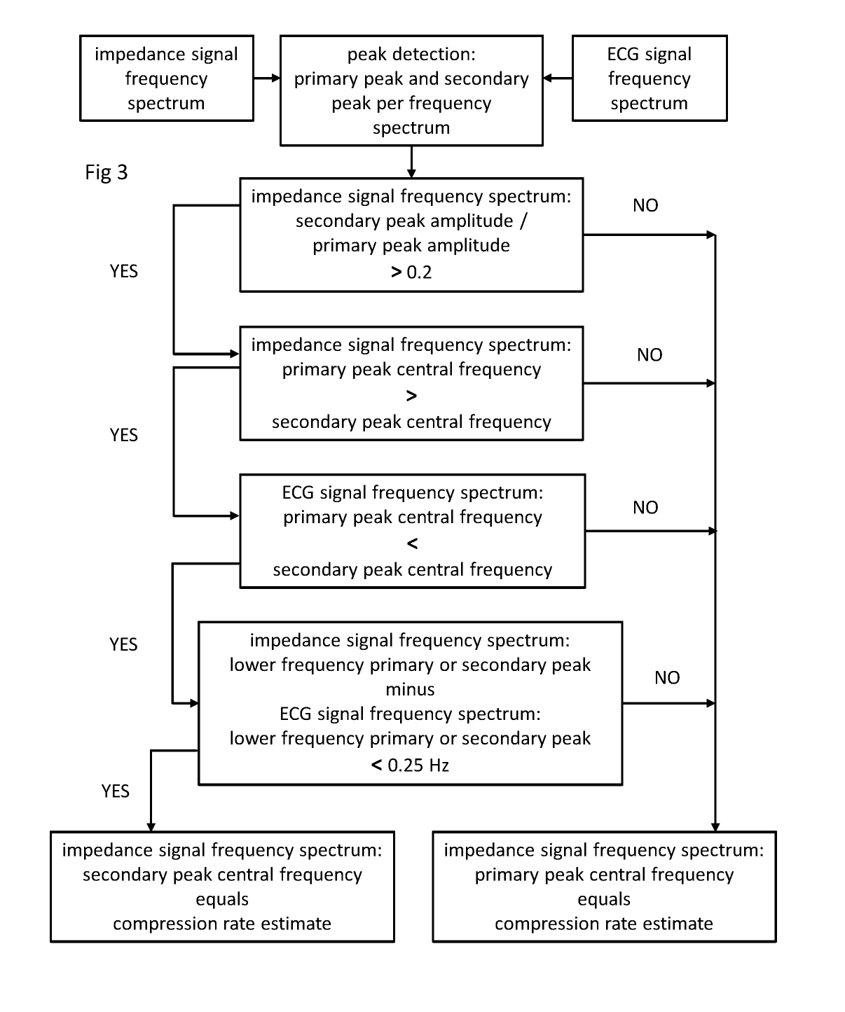

Referring to FIG. 3, in this embodiment, the compression rate estimate processing system 13 of the defibrillator 1 applies four criteria to the impedance signal features and the ECG signal features and compliance is used to select one of the impedance signal compression rate estimates as the true CPR compression rate.

The first criterion applied by the compression rate estimate processing system 13 includes in the impedance signal frequency spectrum a ratio of a secondary peak amplitude and a primary peak amplitude being greater than a pre-determined impedance signal amplitude ratio threshold of 0.2. It will be appreciated that other impedance signal amplitude ratio thresholds may be used, such as, for example, thresholds in the range of 0.05 to 0.5, as well as other values outside of this range.

The second criterion applied by the compression rate estimate processing system 13 includes in the impedance signal frequency spectrum a central frequency of a primary peak being greater than a central frequency of a secondary peak. The third applied by the compression rate estimate processing system 13 criterion includes in the ECG signal frequency spectrum a central frequency of a primary peak being less than a central frequency of a secondary peak. The fourth criterion applied by the compression rate estimate processing system 13 includes a frequency difference between a lower frequency primary or secondary peak in the impedance signal frequency spectrum and a lower frequency primary or secondary peak in the ECG signal frequency spectrum being less than a pre-determined frequency difference threshold of 0.25 Hz. It will be appreciated that other frequency difference thresholds may be used, such as in a range between 0.05 and 0.5 Hz, or other values as well.

The compression rate estimate processing system 13 uses compliance with each of the first to fourth criteria to select an impedance signal compression rate estimate including a central frequency of the secondary peak of the impedance signal frequency spectrum as the CPR compression rate, as shown in the figure. Non-compliance with any of the first to fourth criteria is used by the compression rate estimate processing system 13 to select an impedance signal compression rate estimate including a central frequency of the primary peak of the impedance signal frequency spectrum as the CPR compression rate, as shown in the figure.

The CPR compression rate thus determined is passed to the output unit 15. This outputs feedback based on the CPR compression rate to the user of the defibrillator 1 including an indication that the CPR compression rate is any of satisfactory, too slow, too fast. Outputting the feedback based on the CPR compression rate to the user of the defibrillator provides the user with an opportunity to improve the quality of CPR and gives an indication of the effectiveness of the compressions.

Another aspect of this disclosure includes coverage for a non-transitory computer-readable storage device. For example, an embodiment can include a computer-readable storage device having instructions for controlling a processor, wherein, when the instructions are executed by the processor, the instructions cause the processor to perform operations including receiving an impedance signal of a subject, receiving an electrocardiogram (ECG) signal of the subject, processing the impedance signal to obtain a plurality of impedance signal compression rate estimates, processing the impedance signal to obtain a plurality of impedance signal features, processing the ECG signal to obtain a plurality of ECG signal features and applying a plurality of criteria to the impedance signal features and the ECG signal features and using compliance or non-compliance with one or more of the criteria to select one of the plurality of impedance signal compression rate estimates as the CPR compression rate.

Whether to practice the method or in connection with the defibrillator embodiment, where necessary, computer components are included within the scope of this disclosure. Such components can include, without limitation, a processor, a bus that communicates data between computer components, an input component, an output component, graphical user interfaces, speech processing or speech related components, multi-modal input components, various modules which include computer code programmed to cause the processor to perform certain functions as disclosed herein, or non-transitory computer-readable devices that store computer code or computer-implemented instructions, which, when implemented, cause a processor or a specific module to perform certain operations.

I claim:

1. A defibrillator for determining a rate of cardiopulmonary resuscitation compressions on a subject, comprising:
electrodes adapted to be attached to the subject;
an impedance signal measurement system connected to the electrodes and configured to measure at least one impedance signal of the subject to yield a measured impedance signal;
an electrocardiogram signal measurement system connected to the electrodes and configured to measure an electrocardiogram signal of the subject to yield a measured electrocardiogram signal;
an impedance signal processing system connected to the impedance signal measurement system and configured to process the measured impedance signal of the subject to obtain a plurality of impedance signal compression rate estimates and a plurality of impedance signal features;
an electrocardiogram signal processing system connected to the electrocardiogram signal measurement system and configured to process the measured electrocardiogram signal of the subject to obtain a plurality of electrocardiogram signal features;
a compression rate estimate processing system connected to the impedance signal measurement system and the electrocardiogram signal measurement system and configured to apply a plurality of criteria to the impedance signal features and the electrocardiogram signal features and use compliance with one or more of the criteria to select one of a plurality of impedance signal compression rate estimates as the rate of cardiopulmonary resuscitation compressions; and
an output unit connected to the compression rate estimate processing system and configured to output feedback based on the rate of cardiopulmonary resuscitation compressions to a user of the defibrillator.

2. A defibrillator according to claim 1, in which the impedance signal processing system processes the measured impedance signal to obtain the plurality of impedance signal compression rate estimates by:
using a frequency domain transformation on the measured impedance signal to obtain an impedance signal frequency spectrum;
using a peak detection algorithm to identify a plurality of peaks in the impedance signal frequency spectrum; and
determining central frequencies of the plurality of peaks as the plurality of impedance signal compression rate estimates.

3. A defibrillator according to claim 1, in which the impedance signal processing system processes the measured impedance signal to obtain the plurality of impedance signal features by:
using a frequency domain transformation on the measured impedance signal to obtain an impedance signal frequency spectrum;
using a peak detection algorithm to identify a plurality of peaks in the impedance signal frequency spectrum; and
determining central frequencies and amplitudes of the plurality of peaks as the plurality of impedance signal features.

4. A defibrillator according to claim 1, in which the electrocardiogram signal processing system processes the measured electrocardiogram signal to obtain the plurality of electrocardiogram signal features by:
using a frequency domain transformation on the measured electrocardiogram signal to obtain an electrocardiogram signal frequency spectrum;
using a peak detection algorithm to identify a plurality of peaks in the measured electrocardiogram signal frequency spectrum; and
determining central frequencies and amplitudes of the plurality of peaks as the plurality of electrocardiogram signal features.

5. A defibrillator according to claim 2, in which using the frequency domain transformation to obtain the impedance signal frequency spectrum and the electrocardiogram signal frequency spectrum comprises using any of a Fast Fourier Transform algorithm on a window of the measured impedance signal and a corresponding window of the measured electrocardiogram signal, a Goertzel algorithm on a window of the measured impedance signal and a corresponding window of the measured electrocardiogram signal.

6. A defibrillator according to claim 2, in which using the peak detection algorithm to identify a plurality of peaks in the impedance signal frequency spectrum comprises:
identifying peaks based on decreasing steepness of slopes of the impedance signal frequency spectrum; and
using the peak detection algorithm to identify a plurality of peaks in the electrocardiogram signal frequency spectrum; and
identifying peaks based on decreasing steepness of slopes of the electrocardiogram signal frequency spectrum.

7. A defibrillator according to claim 6, in which the peak detection algorithm returns a primary peak having a highest amplitude at its central frequency, a secondary peak having a next highest amplitude at its central frequency continued up to a specified number of peaks.

8. A defibrillator according to claim 1, in which the impedance signal processing system processes the measured impedance signal to obtain a plurality of additional impedance signal features from the measured impedance signal comprising any of variance of the impedance signal, a morphology of the impedance signal, a gradient of the measured impedance signal, a power of the measured impedance signal, wavelet decomposition of the measured impedance signal, a noise analysis of the measured impedance signal, and a cepstrum of the measured impedance signal.

9. A defibrillator according to claim 1, in which the electrocardiogram signal processing system processes the measured electrocardiogram signal to obtain a plurality of additional electrocardiogram signal features from the electrocardiogram signal comprising any of a variance of the electrocardiogram signal, a morphology of the electrocardiogram signal, a gradient of the electrocardiogram signal, a power of the electrocardiogram signal, a wavelet decomposition of the measured electrocardiogram signal, a noise analysis of the measured electrocardiogram signal, and a cepstrum of the measured electrocardiogram signal.

10. A defibrillator according to claim 1, in which the plurality of criteria applied by the compression rate estimate processing system comprises a criterion comprising in an impedance signal frequency spectrum a ratio of a secondary peak amplitude and a primary peak amplitude being greater than a pre-determined impedance signal amplitude ratio threshold.

11. A defibrillator according to claim 1, in which the plurality of criteria applied by the compression rate estimate processing system comprises a criterion comprising in an impedance signal frequency spectrum a central frequency of a primary peak being greater than a central frequency of a secondary peak.

12. A defibrillator according to claim 1, in which the plurality of criteria applied by the compression rate estimate processing system comprises a criterion comprising in the electrocardiogram signal frequency spectrum a central frequency of a primary peak being less than a central frequency of a secondary peak.

13. A defibrillator according to claim 1, in which the plurality of criteria applied by the compression rate estimate processing system comprises a criterion comprising a frequency difference between a lower frequency primary or secondary peak in an impedance signal frequency spectrum and a lower frequency primary or secondary peak in the electrocardiogram signal frequency spectrum being less than a pre-determined frequency difference threshold.

14. A defibrillator according to claim 1, in which the compression rate estimate processing system uses compliance with one or more of the plurality of criteria to select an impedance signal compression rate estimate as a CPR compression rate.

15. A defibrillator according to claim 1, in which the compression rate estimate processing system uses compliance with each of the plurality of criteria to select an impedance signal compression rate estimate comprising a central frequency of a secondary peak of an impedance signal frequency spectrum as a CPR compression rate.

16. A defibrillator according to claim 1, in which the compression rate estimate processing system uses non-compliance with any of the plurality of criteria to select an impedance signal compression rate estimate comprising a central frequency of a primary peak of an impedance signal frequency spectrum as a CPR compression rate.

17. A defibrillator according to claim 1, in which the plurality of criteria applied by the compression rate estimate processing system comprises a criterion comprising in the electrocardiogram signal frequency spectrum a ratio of a secondary peak amplitude and a primary peak amplitude being greater than a pre-determined electrocardiogram signal amplitude ratio threshold.

18. A defibrillator according to claim 17, in which the compression rate estimate processing system uses compliance with each of the plurality of criteria to select an impedance signal compression rate estimate comprising a central frequency of a secondary peak of an impedance signal frequency spectrum as a CPR compression rate.

19. A defibrillator according to claim 17, in which the compression rate estimate processing system uses non-compliance with any of the plurality of criteria to select an impedance signal compression rate estimate comprising a central frequency of a primary peak of the impedance signal frequency spectrum as a CPR compression rate.

20. A defibrillator according to claim 1, which uses substantially all of the measured impedance signal and substantially all of the measured electrocardiogram signal to determine a CPR compression rate.

21. A defibrillator according to claim 1, which uses one or more portions of the measured impedance signal and corresponding one or more portions of the measured electrocardiogram signal to determine a CPR compression rate.

22. A defibrillator according to claim 21, in which each portion of the measured impedance signal comprises portions which have been pre-analysed for CPR compression rate, which compression rate has not exceeded a threshold used to determine if a rate is a true rate.

23. A defibrillator according to claim 1, in which the output unit outputs feedback based on a CPR compression rate to the user of the defibrillator comprising an indication that the CPR compression rate is any of satisfactory, too slow, too fast.

24. A method of determining a cardiopulmonary resuscitation (CPR) compression rate, the method comprising:
receiving an impedance signal of a subject;
receiving an electrocardiogram signal of the subject;
processing the impedance signal to obtain a plurality of impedance signal compression rate estimates;
processing the impedance signal to obtain a plurality of impedance signal features;
processing the electrocardiogram signal to obtain a plurality of electrocardiogram signal features; and
applying a plurality of criteria to the impedance signal features and the electrocardiogram signal features and using compliance or non-compliance with one or more of the criteria to select one of the plurality of impedance signal compression rate estimates as the CPR compression rate.

25. A computer-readable storage device, having instructions for controlling a processor, wherein, when executed by the processor, causes the processor to perform operations comprising:
receiving an impedance signal of a subject;
receiving an electrocardiogram signal of the subject;
processing the impedance signal to obtain a plurality of impedance signal compression rate estimates;
processing the impedance signal to obtain a plurality of impedance signal features;
processing the electrocardiogram signal to obtain a plurality of electrocardiogram signal features; and
applying a plurality of criteria to the impedance signal features and the electrocardiogram signal features and using compliance or non-compliance with one or more of the criteria to select one of the plurality of impedance signal compression rate estimates as the CPR compression rate.
